(19) United States
(12) Patent Application Publication
(10) Pub. No.: US 2011/0152962 A1
(43) Pub. Date: Jun. 23, 2011

(54) Cable Detection System

(75) Inventors: Michael D. Behm, Chisago City, MN (US); Paul T. Simonette, Maple Lake, MN (US)

(73) Assignee: MEDTRONIC, INC., Minneapolis, MN (US)

(21) Appl. No.: 12/643,213

(22) Filed: Dec. 21, 2009

(51) Int. Cl. A61N 1/362; G01R 19/00; A61B 18/04

(52) U.S. Cl. 607/10; 324/66; 606/33

(57) ABSTRACT

An embodiment of a cable detection system described in this application comprises at least one cable connector adapted to connect with a cable, a field generator adapted to induce an electrical signal across the at least one cable connector, the electrical signal having an amplitude and a frequency, and a detector for detecting a change in the amplitude or the frequency of the electrical signal across the at least one cable connector.

### BACKGROUND

The invention relates generally to electrical devices and more particularly to systems for detecting whether cables are connected to a device and the type of cable connected to a device.

Electronic equipment often uses cables to attach to other electronic equipment or to attach to transducers or other such electrodes, sensors or devices. In order to monitor such equipment and verify proper operation, it is sometimes required to detect that required cables have been attached to the device. It is also sometimes required to detect the type or types of cables that have been attached to the device.

Known cable interconnection systems typically include one or more conductors that are dedicated to the cable detection function. Other systems use a mechanical switch or sensor to determine the presence of a cable. Such systems introduce complexity into the cable connection systems and are not typically easily retrofitted into existing cable connectors.

### SUMMARY

In one embodiment, the invention provides a cable detection system. The cable detection system comprises at least one cable connector adapted to connect with a cable, a field generator adapted to induce an electrical signal across the at least one cable connector, the electrical signal having an amplitude and a frequency, and a detector for detecting a change in the amplitude or the frequency of the electrical signal across the at least one cable connector.

In another embodiment, the invention provides a medical electrical stimulator. The medical electrical stimulator comprises a housing, a circuit board comprising pulse generator modules within the housing and a cable detection system within the housing and coupled to the circuit board comprising at least one cable connector adapted to connect with a cable, a field generator adapted to induce an electrical signal across the at least one cable connector, the electrical signal having an amplitude and a frequency, and a detector for detecting a change in the amplitude or the frequency of the electrical signal across the at least one cable connector.

### BRIEF DESCRIPTION OF THE DRAWINGS

FIG. 1 illustrates a partial perspective view of a connector block assembly utilizing a cable detection system described herein;

FIG. 2 illustrates an enlarged cross-section view of the cable connector shown in FIG. 1;

FIG. 3 illustrates a cable for connecting to the cable connector; and

FIG. 4 is a block diagram illustrating one embodiment of a circuit.

### DETAILED DESCRIPTION

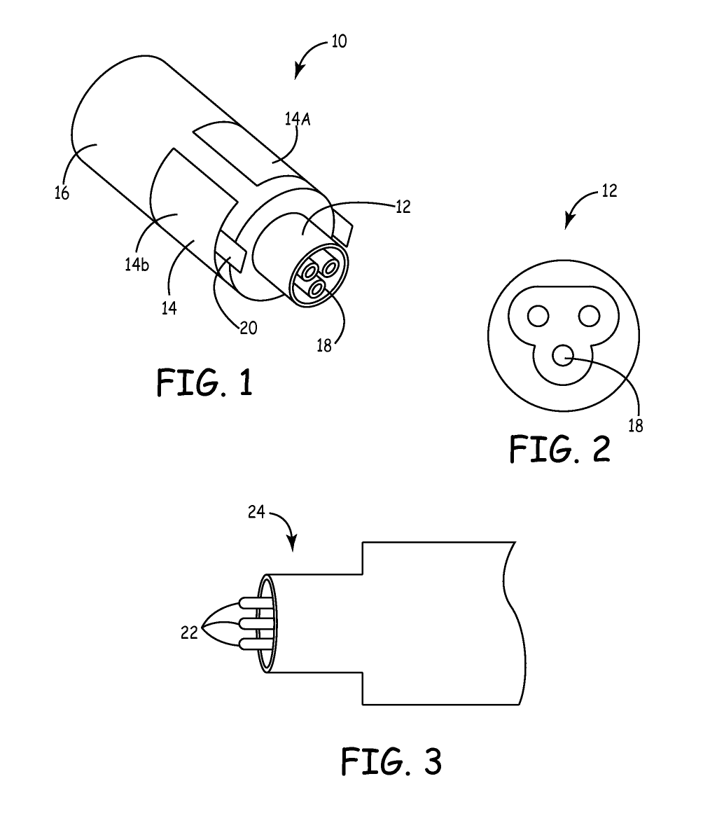

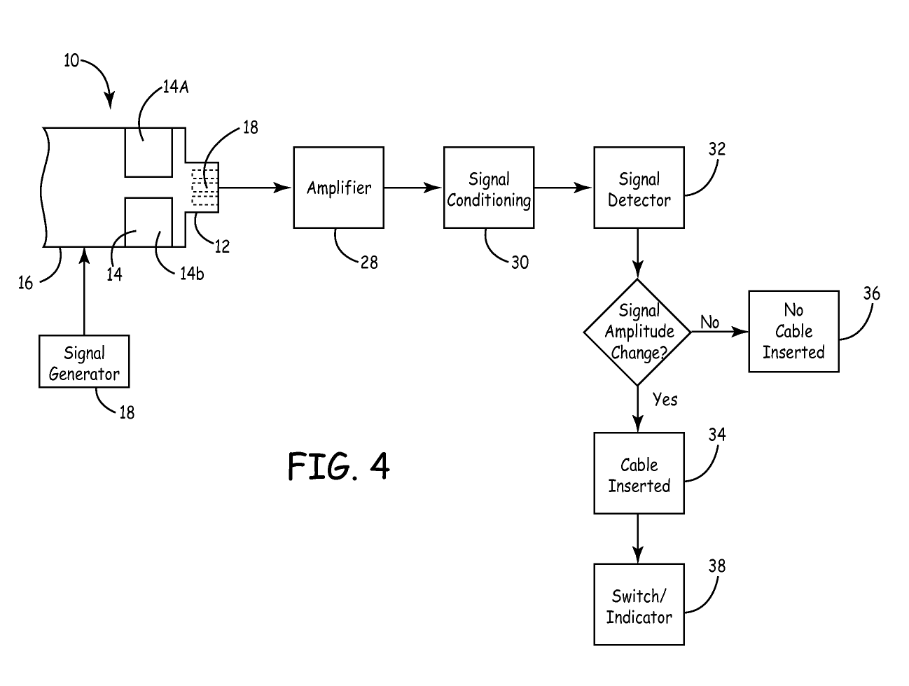

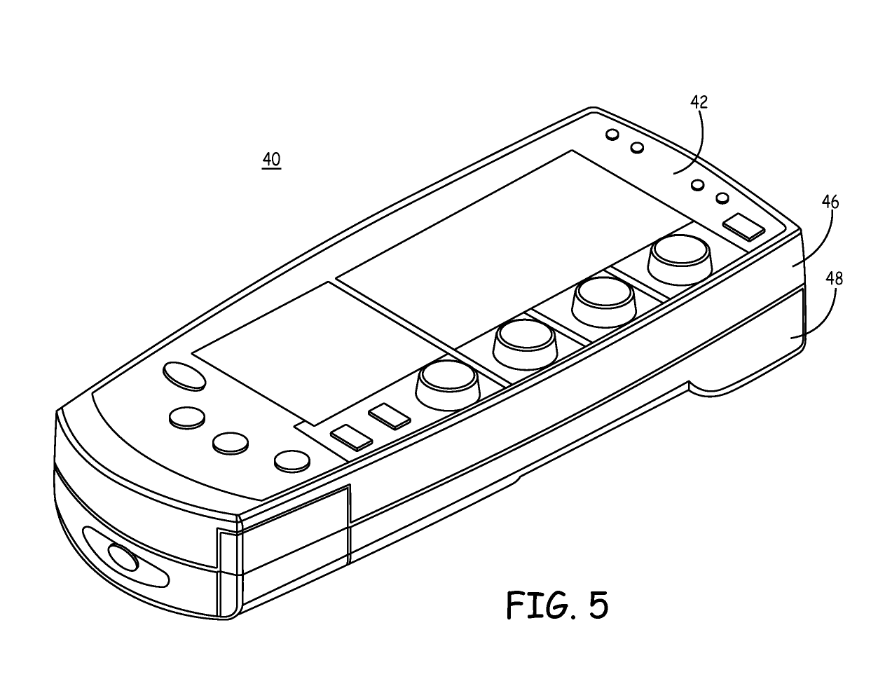

An embodiment of a connector block assembly 10 utilizing an embodiment of a cable detection system of the invention is shown in FIG. 1. In one embodiment, connector block assembly 10 comprises a connector housing 16 coupled to a cable connector 18. On the housing 16 and proximate the cable connector is a field generator 14. The cable connector 12 comprises cable pin receptors 18 for coupling connector pins 24 of cable 26 (see FIG. 3). FIG. 2 is front view of the cable connector 12 and its cable pin receptors 18. The cable pin receptors 18 are typically made from a conductive material.

Connector block assembly 10 as depicted in FIG. 1 is generally cylindrically shaped. However, the connector block assembly could also be any other shape in order to accommodate space requirements within a device. The housing 16 is typically made of a nonconductive material such as polymeric materials.

In this embodiment, the field generator 14 comprises shaped metal sheets or plates 14a and 14b attached to the outside of the connector housing. In this embodiment, the field generator comprises two plates 14a and 14b of a conductive material, for example a metal, having tabs 20 proximate the cable connector. Suitable metals for use in the field generator include copper, tin, nickel, steel, gold, aluminum, tungsten, iron, indium, iridium, magnesium, platinum, carbon, silicon, palladium, silver and combinations and alloys thereof. In other embodiments, the field generator may be located within the connector housing or located near the cable connector. The field generator location is not limited as long as the field generator is capable of inducing an electromagnetic field across the cable connector.

Referring now to FIGS. 2 and 3, cable pin receptors 18 are made from a conductive material, as described above, and are adapted to connect with the conductive connector pins 22 of a cable 24 and form an electrical connection. Typically, the connector pins 22 frictionally engage with the cable pin receptors 18. The cable connector 12 typically also has connection features such as a specific orientation so that the cable is properly connected and a cable securing feature such as a set screw or other locking closure that prevents the cable from disconnecting with the cable connector 12. The cable pin receptors are typically made from the same materials mentioned above for the connector pins and the field generators.

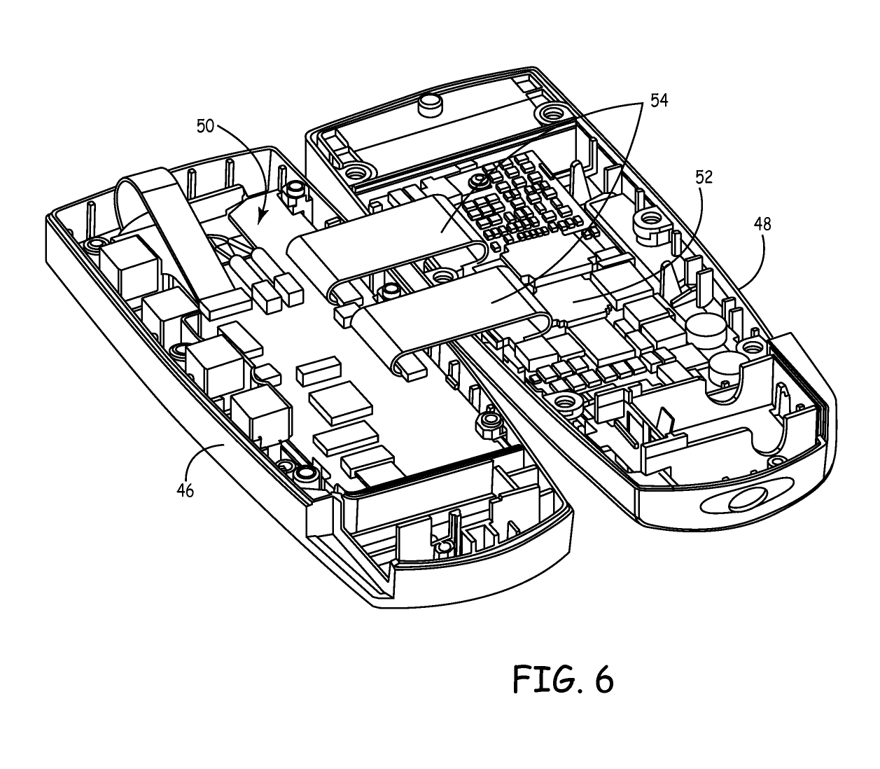

A block diagram of one embodiment of a circuit implementation of a cable detection system of the invention is shown in FIG. 4. Connector block assembly 10 comprises a connector housing 16, a cable connector 12 comprising connector pin receptors 18 coupled to the connector housing 16, and a field generator 14 attached to the outside of the connector housing 16.

In operation, the field generator 14 induces an electromagnetic field from an applied signal from a signal generator 15. Typically, the signal generator generates electromagnetic energy to create the electromagnetic field. The electromagnetic field induces an alternating electrical signal on the connector pin receptors 18, that is, the connector pin receptors function as a receiving antenna. In this embodiment, the electrical signal is amplified through an amplifier 28 and passed through a signal conditioner 30 that converts the alternating current (AC) to an electrical signal that can be sampled by the detector. When the detector 32 detects a change in the amplitude of the electrical signal, for example, voltage (V) or current (A), or a change in the frequency (Hz) of the indication is that a cable is inserted 34. The capacitance of the inserted cable reduces the amplitude of the electrical signal or the frequency of the electromagnetic field. If the detector detects no change in the amplitude or the frequency of the electrical signal, the indication is that no cable is inserted 36. For example, the detected rectified DC signal without a cable inserted was about 3 volts. After the cable was inserted, the detected rectified DC signal was less than 1 volt, that is, the resulting amplitude was about 33% of the amplitude with no cable inserted or a decrease in the amplitude of the electrical signal before cable insertion of about 67%. In other embodiments, the resulting amplitude may range from about 10% to about 90% of the amplitude of the electrical signal prior to insertion of a cable.

Other useful detectors that can be used in the cable detection system of the invention include those that detect a frequency shift, a phase shift, changes in AC or DC current and a peak detector.

Once a cable connection is detected by the cable detection system, a switch and/or indicator 38 can be triggered to communicate or indicate information about the cable, for example, the type of cable used and/or mode of the device to a user. For example, once the type of cable is identified, a switch can be configured to direct a device capable of operating in different modes to select an operating mode based on the identity or type of cable inserted.

Turning to FIGS. 5 and 6, they depict, in exterior and interior views, the major components of a medical electrical stimulator 40, specifically a medical device pulse generator operating system with external controls and LCD panel displays for adjusting and displaying the operating mode and parameters of operation thereof, in which the present invention may be implemented. The stimulator 40 encases the pulse generator circuit within a housing 42 comprising an upper case 46 and a lower case 48. The interior of the upper case 46 supports a user interface module 50 including the LCD display panels, rotary dials and keys, shown in FIG. 5 exposed on or through the faceplate of the upper case 46, to be manipulated by the user to operate the pulse generator. The interior of the lower case 48 supports a circuit board 52 on which the pulse generator modules are assembled, the pulse generator modules including a timing and control module, an external communications module, a power supply module, a sense amp module, and a pacing output module. Flexible printed circuits 54 interconnect the pulse generator modules on circuit board 52 with the user interface module 50. The user interface module 50 and pulse generator modules mounted on printed circuit board 52, as well as the operation of the medical device pulse generator, are disclosed in detail in U.S. Pat. No. 5,626,621. In one embodiment, the medical electrical stimulator is capable of operating in multiple single and dual chamber modes, for example, AAI, VVI and DDD or DDI modes.

Cable detection systems described in this application can be used in a wide variety of devices. Examples of such devices include medical devices, such as electrocardiographs, external pacemakers, RF generators, cauterizing devices, and sensors; communications devices; and consumer electronics.

The embodiments and the examples described herein are exemplary and not intended to be limiting in describing the full scope of apparatus, systems, and methods of the present technology. Equivalent changes, modifications and variations of some embodiments, materials, compositions and methods can be made within the scope of the present technology, with substantially similar results.

What is claimed is:

1. A cable detection system comprising:
at least one cable connector adapted to connect with a cable;
a field generator adapted to induce an electrical signal across the at least one cable connector, the electrical signal having an amplitude and a frequency; and
a detector for detecting a change in the amplitude or the frequency of the electrical signal across the at least one cable connector.

2. The cable detection system of claim 1 wherein the detector comprises an antenna and a detector circuit coupled to the antenna.

3. The cable detection system of claim 1 wherein the antenna comprises pin receptors.

4. The cable detection system of claim 1 wherein the field generator is a radio frequency signal generator.

5. The cable detection system of claim 1 wherein the at least one cable connector further comprises a cable connector housing and wherein the field generator is located on the cable connector housing.

6. The cable detection system of claim 1 wherein the at least one cable connector further comprises a cable connector housing wherein the field generator comprises electrical conductors located on the cable connector housing.

7. The cable detection system of claim 1 wherein the at least one cable connector further comprises a cable connector housing wherein the signal generator comprises field electrical conductors located inside of the cable connector housing.

8. The cable detection system of claim 1 wherein the field generator comprises electrical conductors located near the cable connector.

9. The cable detection system of claim 1 further comprising a cable connected to the at least once cable connector and a reduction in amplitude of the electrical signal across the at least one cable connector.

10. The cable detection system of claim 9 wherein the reduction in amplitude is about 67%.

11. A medical electrical stimulator comprising:
a housing;
a circuit board comprising pulse generator modules within the housing; and
a cable detection system within the housing comprising at least one cable connector adapted to connect with a cable;
a field generator adapted to induce an electrical signal across the at least one cable connector, the electrical signal having an amplitude and a frequency; and
a detector for detecting a change in the amplitude or the frequency of the electrical signal across the at least one cable connector.

12. The medical electrical stimulator of claim 11 further comprising a cable connected to the at least once cable connector and a reduction in amplitude of the electrical signal across the at least one cable connector.

13. The medical electrical stimulator of claim 11 wherein the medical electrical stimulator is an external pacemaker, an RF generator, or a cauterizing device.

14. The medical electrical stimulator of claim 12 further comprising means for identifying the cable and means for indicating the cable identification to a user.

15. The medical electrical stimulator of claim 14 further comprising a switch capable of selecting an operating mode based on the identity of the cable.
